(19) United States
(12) Patent Application Publication
(10) Pub. No.: US 2021/0402359 A1
(43) Pub. Date: Dec. 30, 2021

(54) TURBULENT FLOW MIXING BAG AND RELATED SYSTEMS AND METHODS

(71) Applicant: Fresenius Medical Care Holdings, Inc., Waltham, MA (US)

(72) Inventors: Jared Henry Lamb, West Haven, UT (US); Carol Phil Beasley, III, Layton, UT (US); Jeffrey Allen Wagner, Sandy, UT (US)

(21) Appl. No.: 17/354,095

(22) Filed: Jun. 22, 2021

Related U.S. Application Data
(60) Provisional application No. 63/043,576, filed on Jun. 24, 2020.

(51) Int. Cl. B01F 15/00; B01F 15/02; B01F 1/00; B01F 5/06; A61M 1/16

(52) U.S. Cl. CPC B01F 15/0085; B01F 15/00889; B01F 15/00772; B01F 15/0212; B01F 2005/0025; B01F 5/0603; A61M 1/167; B01F 2215/0422; B01F 2215/0034; B01F 1/0022

(57) ABSTRACT

The disclosed bag for mixing materials may include a front wall, a back wall, a mixing chamber between the front wall and the back wall, a first sidewall between the front wall and the back wall and defining a first side of the mixing chamber, a second sidewall between the front wall and the back wall defining a second, opposite side of the mixing chamber, and a port positioned to provide fluid access to the mixing chamber from a bottom of the mixing chamber, wherein the first sidewall and the second sidewall are shaped to alternate a direction of fluid flow when fluid is introduced into the mixing chamber through the port. Various other components, systems, and methods are also disclosed.

### CROSS-REFERENCE TO RELATED APPLICATION

This application claims the benefit of U.S. Provisional Patent Application No. 63/043,576, titled “TURBULENT FLOW MIXING BAG AND RELATED SYSTEMS AND METHODS,” filed 24 Jun. 2020, the entire disclosure of which is incorporated herein by reference.

### BACKGROUND

Dialysis is a treatment for patients who have experienced kidney failure. In individuals with fully functioning kidneys, the kidneys remove excess water and nitrogen waste materials (e.g., in the form of urea and creatinine) from the blood and pass these materials to the bladder for expulsion from the body. Without properly functioning kidneys, a patient may not be able to maintain proper blood pH and pressure, electrolyte or fluid balance, among other problems. Dialysis may replace or supplement the kidneys' function in such patients.

Hemodialysis is a form of dialysis in which blood is drawn from a patient via an artery, passed through a dialyzer, and returned to the patient via a vein. The dialyzer includes a semi-permeable membrane with the patient's blood passing along one side of the membrane and a dialysate solution passing (generally countercurrent) on the other side of the membrane. The dialysate solution typically includes an acid and bicarbonate in purified water. Waste products pass from the blood to the dialysate solution and treated (e.g., cleaned) blood can pass out of the dialyzer and back to the patient's circulatory system. The blood is delivered to the dialyzer through tubing and a variety of other components. For example, the blood may pass through a pump for moving the blood through tubing, one or more drip chambers used to ensure no air bubbles are present in the blood passing through the dialyzer or returned to the patient, a pressure sensor, an anti-clotting system, a heater, a blood volume monitor, and potentially various other sensors and systems. In some dialysis machines, the dialysate solution is formed by mixing one or more solid solutes in water.

### BRIEF DESCRIPTION OF THE DRAWINGS

The accompanying drawings illustrate a number of example embodiments and are a part of the specification. Together with the following description, these drawings demonstrate and explain various principles of the present disclosure.

FIG. 1 is a schematic diagram of a hemodialysis system, according to at least one embodiment of the present disclosure.

FIG. 2 is a detailed front view of turbulent flow mixing bag, according to an embodiment of the present disclosure.

FIG. 3 is a detailed front view of turbulent flow mixing bag, according to another embodiment of the present disclosure.

FIG. 4 is a detailed front view of turbulent flow mixing bag, according to another embodiment of the present disclosure.

FIG. 5 is a detailed front view of turbulent flow mixing bag, according to another embodiment of the present disclosure.

FIG. 6 is a detailed front view of turbulent flow mixing bag, according to another embodiment of the present disclosure.

FIG. 7 is an illustration of a fluid flow model showing turbulence in a mixing bag, according to at least one embodiment of the present disclosure.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the present disclosure covers all modifications, equivalents, and alternatives falling within the scope of the appended claims.

### DETAILED DESCRIPTION OF EXAMPLE EMBODIMENTS

The present disclosure is generally directed to turbulent flow mixing bags that may be used for mixing materials with each other, such as for dissolving a solute in a solvent.

In some embodiments, the turbulent flow mixing bags may be useful for dissolving a solid material (e.g., a sodium bicarbonate material) in a fluid (e.g., a water solvent) for hemodialysis, although the turbulent flow mixing bags may be useful in other contexts and applications as well. As will be explained in further detail below, the present disclosure includes a turbulent flow mixing bag for mixing a dialysate solution and related hemodialysis systems that may achieve one or more improvements over conventional methods and devices for mixing dialysate solution for hemodialysis. For example, the turbulent flow mixing bag of the present disclosure may include a front wall, a back wall, a mixing chamber between the front wall and the back wall, a first sidewall between the front wall and the back wall and defining a first side of the mixing chamber, a second sidewall between the front wall and the back wall defining a second, opposite side of the mixing chamber, and a port positioned to provide fluid access to the mixing chamber from a bottom of the mixing chamber. The first sidewall and the second sidewall may be shaped to alternate a direction of fluid flow when fluid is introduced into the mixing chamber through the port and/or when mixed fluid is withdrawn from the mixing chamber through the port. Protrusions and vertices defined by the sidewalls of the mixing bag may alternate the direction of fluid flow and create a turbulent flow of the injected fluid for mixing with the solid solute.

Features from any of the embodiments described herein may be used in combination with one another in accordance with the general principles described herein. These and other embodiments, features, and advantages will be more fully understood upon reading the following detailed description in conjunction with the accompanying drawings and claims.

In some examples, relational terms, such as “first,” “second,” “top,” “bottom,” etc., may be used for clarity and convenience in understanding the disclosure and accompanying drawings and may not necessarily connote or depend on any specific preference, orientation, or order, except where the context clearly indicates otherwise.

In some examples, the term “substantially” in reference to a given parameter, property, or condition may mean and include to a degree that one of ordinary skill in the art would understand that the given parameter, property, or condition is met within a small degree of variance, such as within acceptable manufacturing tolerances. By way of example, depending on the particular parameter, property, or condition that is substantially met, the parameter, property, or condition may be at least 90% met, at least 95% met, at least 99% met, or fully met.

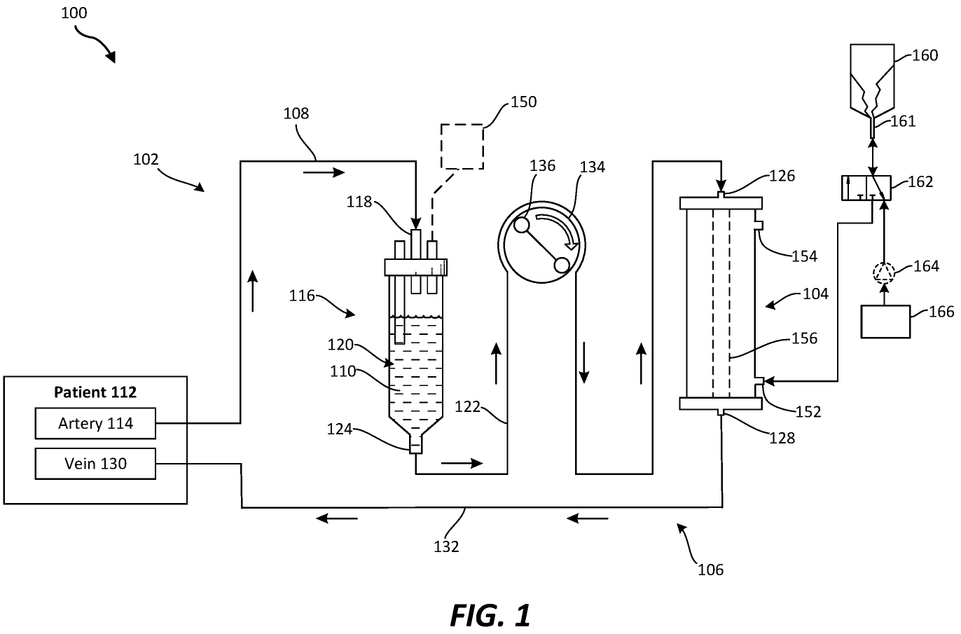

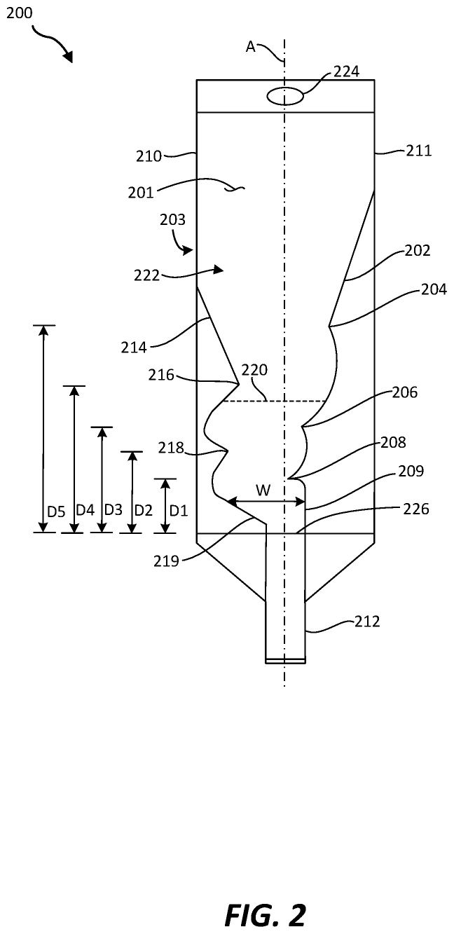

The following will provide, with reference to FIG. 1, detailed descriptions of hemodialysis systems according to embodiments of the present disclosure. With reference to FIGS. 2-6, the following will provide detailed descriptions of turbulent flow mixing bags for use with hemodialysis systems.

FIG. 1 is a schematic diagram of a hemodialysis system 100, according to at least one embodiment of the present disclosure. The hemodialysis system 100 may include an arterial-side tubing set 102, a dialyzer 104, and a venous-side tubing set 106.

The arterial-side tubing set 102 may include a first flexible tube 108 that may be configured to receive blood 110 from a patient 112 (e.g., from an artery 114 of the patient 112). The arterial-side tubing set 102 may also include an arterial chamber 116 (e.g., a drip chamber) with a blood inlet port 118 fluidically coupled to the first flexible tube 108. Thus, the blood inlet port 118 may be configured to receive the blood 110 from the first flexible tube 108 and to convey the blood 110 into an interior 120 of the arterial chamber 116. The arterial-side tubing set 102 may also include a second flexible tube 122. The arterial chamber 116 may be configured to collect air from the blood 110 of the patient 112 as the blood 110 passes through the arterial chamber 116. One end of the second flexible tube 122 may be fluidically coupled to a blood outlet port 124 of the arterial chamber 116 and an opposing end of the second flexible tube 122 may be fluidically coupled to a dialyzer blood inlet 126 of the dialyzer 104. In some embodiments, the arterial-side tubing set 102 may include additional components, such as one or more fluid access ports, pressure sensors, line clamps, etc.

The venous-side tubing set 106 may include at least one third flexible tube 132 fluidically coupled to a dialyzer blood outlet 128 of the dialyzer 104. The third flexible tube 132 may be configured to receive the blood 110 from the dialyzer 104 and to convey the blood 110 (e.g., cleaned blood) back to the patient 112, such as to a vein 130 of the patient 112. In some embodiments, the venous-side tubing set 106 may include additional components, such as one or more fluid access ports, pressure sensors, line clamps, venous chambers (e.g., a venous chamber the same as or similar to the arterial chamber 116), etc.

Each of the first flexible tube 108, second flexible tube 122, and third flexible tube 132 may include (e.g., may be formed of), without limitation, a medical grade polymer material with hemocompatibility, such as polyvinylchloride (“PVC”), silicone, polytetrafluoroethylene (“PTFE”), etc. The flexible tubes 108, 122, 132 may be substantially transparent to visible light, such as to facilitate identification of flow of the blood 110, potential clotting, and/or potential air bubbles. Each of the flexible tubes 108, 122, 132 may include a single section of continuous tubing or may include two or more connected segments of tubing.

A pump 134 may be positioned and configured to force the blood 110 through the arterial-side tubing set 102, dialyzer 104, and venous-side tubing set 106. As illustrated in FIG. 1, the pump 134 may be positioned downstream from the arterial chamber 116 and may be operatively coupled to the second flexible tube 122. In this configuration, the arterial chamber 116 may be in a pre-pump position. The pump 134 may apply (e.g., through a portion of the second flexible tube 122) a negative pressure to the blood outlet port 124 of the arterial chamber 116 to draw the blood 110 through the arterial chamber 116 from the blood inlet port 118 to the blood outlet port 124. In some embodiments, the pump 134 may be a roller-type pump that includes one or more rollers 136 for rolling along a section of the second flexible tube 122 to progressively compress the second flexible tube 122 and to draw the blood 110 through the arterial-side tubing set 102 (and ultimately also through the dialyzer 104 and venous-side tubing set 106) at a controlled flow rate. In some examples, the section of the second flexible tube 122 that interacts with the pump 134 may have a larger diameter than other sections of the second flexible tube 122. In addition, the section of the second flexible tube 122 that interacts with the pump 134 may be formed of a different material relative to other sections of the second flexible tube 122, such as to exhibit mechanical properties that are suitable for interacting with the pump 134 (e.g., for being repeatedly compressed by the rollers 136).

In some embodiments, an auxiliary element 150 may be connected to the arterial chamber 116. By way of example and not limitation, the auxiliary element 150 may be a saline solution source, an anticoagulant (e.g., heparin) source, a pressure sensor, an air release valve, a medication source, etc.

In some examples, the dialyzer 104 may include the dialyzer blood inlet 126, the dialyzer blood outlet 128, a dialysate inlet 152, a dialysate outlet 154, and a semi-permeable membrane 156. As noted above, the dialyzer blood inlet 126 may be fluidically coupled to the arterial-side tubing set 102 for receiving the blood 110 from the patient 112. The dialyzer blood outlet 128 may be fluidically coupled to the venous-side tubing set 106 for returning the blood 110 (e.g., cleaned blood) to the patient 112. The dialysate inlet 152 may be configured for flowing a dialysate solution into the dialyzer 104 and the dialysate outlet 154 may be configured for flowing the dialysate solution and waste products from the blood 110 out of the dialyzer 104. The semi-permeable membrane 156 may be positioned within the dialyzer 104 and may physically separate at least a portion of the blood 110 (e.g., blood cells) from the dialysate solution while allowing waste products (e.g., urea, etc.) from the blood 110 to pass through the semi-permeable membrane 156 to be withdrawn from the dialyzer 104 with the dialysate solution through the dialysate outlet 154.

The dialysate solution and the blood 110 may flow in a countercurrent fashion to enhance the transfer of waste products from the blood 110 to the dialysate solution. As shown in FIG. 1, for example, the blood 110 may flow downward through the dialyzer 104 and the dialysate solution may flow upward through the dialyzer 104. In additional examples, the blood 110 may flow upward through the dialyzer 104 and the dialysate solution may flow downward through the dialyzer 104.

In some examples, the dialyzer 104 may receive the dialysate solution from a mixing bag 160. The dialysate solution may be mixed in the mixing bag 160 as described in detail below with reference to FIGS. 2-7. The dialysate solution in the mixing bag 160 may flow through a valve 162 and the dialysate inlet 152 into the dialyzer 104. The valve 162 may include three ports and may operate at two positions. For example, the valve 162 may include a first port fluidically coupled to a mixing bag port 161, a second port fluidically coupled to the dialysate inlet 152, and a third port coupled to a fluid source 166. In some examples, a pump 164 may be fluidically coupled between the third port of the valve 162 and the fluid source 166. The valve 162 may operate at a first position in which the third port of the valve 162 allows fluid (e.g., purified water) to flow from the fluid source 166 through the mixing bag port 161 into the mixing bag 160 while the second port fluidically coupled to the dialysate inlet 152 is blocked. In one example, the fluid may be gravity-fed through the valve 162. In addition or alternatively, the pump 164 may pump the fluid from the fluid source 166 through a fluid conduit (e.g., a tube) into the mixing bag port 161 and into the mixing bag 160. The fluid may be mixed with a solid solute in mixing bag 160 to form a dialysate solution using any suitable method. For example, the fluid may be mixed with the solid solute in the mixing bag 160 by inducing a turbulent flow of the fluid in the mixing bag 160 as described in detail below with reference to FIGS. 2-7. The valve 162 may operate at a second position in which the first port fluidically coupled to the mixing bag port 161 allows fluid to flow to the second port fluidically coupled to the dialysate inlet 152 while the third port fluidically coupled to the fluid source 166 is blocked.

While FIG. 1 shows a single mixing bag 160 in the hemodialysis system 100 by way of illustration, the present disclosure is not so limited. In additional embodiments, two or more mixing bags 160 may be present to form different components of the dialysate. For example, multiple mixing bags 160 may be employed to respectively mix a fluid (e.g., purified water) with various solid solutes, such as bicarbonate, electrolyte(s), sodium chloride, and/or dextrose, etc.

FIG. 2 is a detailed front view of a turbulent flow mixing bag 200, according to an embodiment of the present disclosure. The turbulent flow mixing bag 200 may be configured to mix materials (e.g., a liquid solvent with a solid solute) based on a turbulent flow of a fluid (e.g., a liquid) injected into the mixing bag 200. The mixing bag 200 may include a front wall 201, a back wall 203, and a mixing chamber 222 between the front wall 201 and the back wall 203. The materials may be mixed in the mixing chamber 222 by the turbulent fluid motion of the injected fluid. The fluid motion may include chaotic and/or random changes in pressure and flow velocity within the mixing chamber 222 that may cause the injected fluid to mix with one or more materials (e.g., a solid solute, such as bicarbonate, an electrolyte, sodium chloride, dextrose, etc.) within the mixing chamber 222.

The mixing bag 200 may include a first sidewall 210 between the front wall 201 and the back wall 203. The first sidewall 210 may define a first side of the mixing chamber 222. The mixing bag 200 may also include a second sidewall 211 between the front wall 201 and the back wall 203 defining a second, opposite side of the mixing chamber 222. The front wall 201, back wall 203, first sidewall 210, and second sidewall 211 may include a polymer material (e.g., a medical grade polymer, polyvinyl chloride, polypropylene, copolyester ether, polyolefin, etc.). The mixing bag 200 may also include a port 212 fluidically coupled to the mixing chamber 222. The port 212 may be positioned and configured to provide fluid access to the mixing chamber 222 from a bottom of the mixing chamber 222. As will be described in further detail below, the first sidewall 210 and second sidewall 211 may be shaped and configured to alternate a direction of fluid flow when fluid is introduced into the mixing chamber 222 through the port 212 and/or when fluid is withdrawn from the mixing chamber 222 through the port 212 to create the turbulent flow for mixing the materials.

In some examples, the first sidewall 210 may include a plurality of straight sections 214, 219 that may be oriented at non-parallel angles to a longitudinal axis A of the mixing chamber 222. Additionally or alternatively, the second sidewall 211 may include a plurality of straight sections 202, 209 that may be oriented at non-parallel angles to the longitudinal axis A of the mixing chamber 222. Although FIG. 2 shows each of the first sidewall 210 and the second sidewall 211 as including two straight sections of sidewalls, the present disclosure is not so limited. Rather, the sidewalls 210, 211 of the mixing bag 200 may include any number of straight sections. As shown in FIG. 2, in some embodiments the angles of the straight sections 214, 219 of the first sidewall 210 may be different from the angles of the straight sections 202, 209 of the second sidewall 211. In some examples, each of the angles of the straight sections 214, 219 of the first sidewall 210 and the straight sections 202, 209 of the second sidewall 211 may be between about 20 degrees and about 80 degrees from the longitudinal axis. Different angles for each of the straight sections 202, 209, 214, 219 may contribute to inducing turbulence for mixing the materials within the mixing chamber 222.

In some examples, a lateral width W, taken perpendicular to the longitudinal axis A of the mixing bag 200, between the first sidewall 210 and the second sidewall 211 may generally increase as a distance from the port 212 increases. In some examples, the lateral width W may change between an increasing width and a decreasing width as a distance from the port 212 increases. Changing the lateral width W between first and second sidewalls 210, 211 may affect the turbulent flow for improved mixing of the materials (e.g., a solvent and a solid solute). The lateral width W distant from the port 212 may be generally greater than the lateral width W proximate to the port 212, such as to improve general downward flow of the materials as the mixing chamber 222 is drained.

The mixing bag 200 may be manufactured to define the straight, angled, and curved sections of the first sidewall 210 and the second sidewall 211. For example, manufacturing the mixing bag 200 may include, without limitation, performing heat sealing, radio frequency sealing, hot bar welding, adhering, or a combination thereof to the front wall 201 and the back wall 203. In some examples, a mold that replicates the straight, angled, and curved sections of the first sidewall 210 and second sidewall 211 may be used to seal the front wall 201 and the back wall 203 together to form the shape of the mixing chamber 222.

The mixing bag 200 may also include a hanger feature 224 at an end (e.g., a top) of the mixing bag 200 opposite the port 212. The hanger feature 224 may be configured for hanging the mixing bag 200 to support the mixing bag 200. For example, the hanger feature 224 may include a hole or a slit for hanging the mixing bag 200 on a support pole.

The mixing bag 200 may also include a membrane 226 initially covering the port 212, such as to inhibit the loss of solid solute through the port 212 prior to mixing the solid solute with a fluid. The membrane 226 may be configured to break when a sufficient fluid pressure is applied to the membrane 226, to enable the fluid to flow through the port 212 into the mixing chamber 222.

In some examples, the mixing chamber 222 may be sized and configured for holding a solid solute and for at least partially dissolving the solid solute in a liquid solvent (e.g., a fluid, purified water, etc.) entering the mixing chamber 222 through the port 212. The solid solute may partially fill the mixing chamber 222 before introduction of the fluid. For example, the solid solute may fill the mixing chamber 222 from the port 212 to a fill line 220.

When the liquid solvent (e.g., a fluid, purified water, etc.) is injected into the port 212, the membrane 226 may burst from the fluid pressure and allow the fluid to flow into the mixing chamber 222. The fluid may be injected from the fluid source 166 through valve 162 of FIG. 1. The injected fluid may flow into the mixing chamber 222 to mix with the solid solute. The mixing chamber 222 may include protrusions 204, 206, 208, 216, and 218 along the first sidewall 210 and the second sidewall 211 of the mixing chamber 222. The protrusions 204, 206, and 208 may be features of the second sidewall 211. The protrusions 216 and 218 may be features of the first sidewall 210. In some examples, each of the protrusions may include a vertex extending laterally into the mixing chamber 222. The vertex of each of the protrusions 204, 206, 208, 216, and 218 may be located at a distance from a lower end of the mixing chamber 222. For example, as illustrated in FIG. 2, the vertex of the protrusion 204 may be located a distance D5 from the lower end of the mixing chamber 222, the vertex of the protrusion 206 may be located a distance D3 from the lower end of the mixing chamber 222, the vertex of the protrusion 208 may be located a distance D1 from the lower end of the mixing chamber 222, the vertex of the protrusion 216 may be located a distance D4 from the lower end of the mixing chamber 222, and the vertex of the protrusion 218 may be located a distance D2 from the lower end of the mixing chamber 222. By way of example and not limitation, the distance D1 may be between about 15 mm and about 35 mm (e.g., about 28 mm), the distance D2 may be greater than the distance D1 and may be between about 35 mm and about 55 mm (e.g., about 45 mm), the distance D3 may be greater than the distance D2 and may be between about 55 mm and about 80 mm (e.g., about 68 mm), the distance D4 may be greater than the distance D3 and may be between about 80 mm and about 110 mm (e.g., about 91 mm), and the distance D5 may be greater than the distance D4 and may be between about 110 mm and about 150 mm (e.g., about 130 mm). The distances D1-D5 may each be adjusted to alter the turbulence and other flow characteristics of fluid within the mixing bag 200. Additionally, mixing bags 200 of different sizes (e.g., larger or smaller than the mixing bag 200 shown in FIG. 2) may have protrusions 204, 206, 208, 216, and 218 at respectively larger or smaller distances D1-D5.

In some examples, as the fluid flows into the mixing chamber 222, the fluid passes by the vertices of the protrusions 204, 206, 208, 216, and 218, resulting in turbulence in the fluid flow. The turbulence in the fluid flow may create vortices which interact with each other and interact with the solid solute. The kinetic energy in the turbulent fluid flow may accelerate the homogenization (e.g., mixing) of the fluid solvent and the solid solute. The homogenization of the fluid and the solid solute may be based on factors including, without limitation, the pressure and/or flow rate of the fluid entering port 212, the location of the protrusions 204, 206, 208, 216, and 218, the number of protrusions 204, 206, 208, 216, and 218, the shape of the protrusions 204, 206, 208, 216, and 218, the distance between the protrusions 204, 206, 208, 216, and 218, and the viscosity of the fluid. The interaction between the turbulent fluid and the solid solute may mix the fluid and the solid solute more thoroughly than a mixing chamber lacking the protrusions 204, 206, 208, 216, and 218.

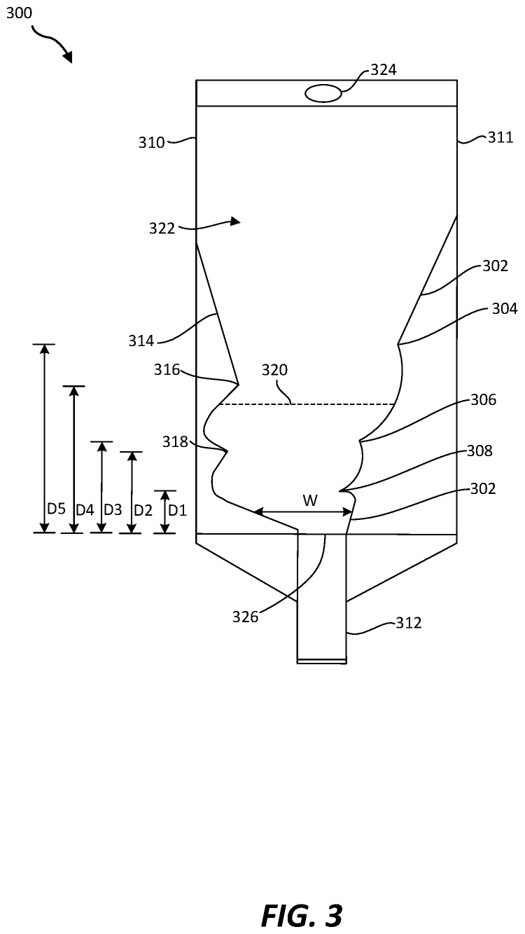

FIG. 3 is a detailed front view of a turbulent flow mixing bag 300, according to another embodiment of the present disclosure. The turbulent flow mixing bag 300 may be configured to mix materials (e.g., a liquid solvent and a solid solute) based on a turbulent flow of a fluid (e.g., a liquid) injected into the mixing bag 300. The mixing bag 300 may include a front wall, a back wall, and a mixing chamber 322 between the front wall and the back wall. The materials may be mixed in the mixing chamber 322 by the turbulent fluid motion of the injected fluid. The fluid motion may include chaotic and/or random changes in pressure and flow velocity within the mixing chamber 322 that cause the injected fluid to mix with materials (e.g., a solid solute) within the mixing chamber 322. The mixing bag 300 may include a first sidewall 310 between the front wall and the back wall. The first sidewall 310 may define a first side of the mixing chamber 322. The mixing bag 300 may also include a second sidewall 311 between the front wall and the back wall defining a second, opposite side of the mixing chamber 322. The front wall, back wall, first sidewall 310, and second sidewall 311 may include a polymer material (e.g., a medical grade polymer, polyvinyl chloride, polypropylene, copolyester ether, polyolefin, etc.). The mixing bag 300 may include a port 312 fluidically coupled to the mixing chamber 322 that may be positioned and configured to provide fluid access to the mixing chamber 322 from a bottom of the mixing chamber 322. As will be described in further detail below, the first sidewall 310 and second sidewall 311 may be shaped and configured to alternate a direction of fluid flow when fluid is introduced into the mixing chamber 322 through the port 312 to create the turbulent liquid flow for mixing the materials.

In some examples, the first sidewall 310 may include a plurality of straight sections 314, 319 that may be oriented at non-parallel angles to a longitudinal axis of the mixing chamber 322. Additionally or alternatively, the second sidewall 311 may include a plurality of straight sections 302, 309 that may be oriented at non-parallel angles to the longitudinal axis of the mixing chamber 322. Although FIG. 3 shows each of the first sidewall 310 and the second sidewall 311 as including two straight sections of sidewalls, the present disclosure is not so limited and the mixing bag 300 may include any number of straight sections. As shown in FIG. 3, the angles of the straight sections 314, 319 of the first sidewall 310 may be different from the angles of the straight sections 302, 309 of the second sidewall 311. In some examples, each of the angles of the straight sections 314, 319 of the first sidewall 310 and the straight sections 302, 309 of the second sidewall 311 may be between about 20 degrees and about 80 degrees from the longitudinal axis. Different angles for each of the straight sections 302, 309, 314, 319 may contribute to the turbulent liquid flow for mixing the materials.

In some examples, a lateral width W between the first sidewall 310 and the second sidewall 311 may generally increase as a distance from port 312 increases. In some examples, the lateral width W between the first sidewall 310 and the second sidewall 311 may change between an increasing width and a decreasing width as a distance from the port 312 increases. For example, the average lateral width W between the first and second sidewalls 310, 311 of the mixing bag 300 of FIG. 3 may be greater than the average lateral width W between the first and second sidewalls 210, 211 of the mixing bag 200 of FIG. 2. Changing the lateral width W between the first and second sidewalls 310, 311 may affect the turbulence of the materials mixing in the mixing bag 300.

The mixing bag 300 may also include a hanger feature 324 at an end (e.g., a top) of the mixing bag 300 opposite the port 312. The hanger feature 324 may be configured for hanging the mixing bag 300 to support the mixing bag 300. For example, the hanger feature 324 may include a hole or a slit for hanging the mixing bag 300 on a support pole.

The mixing bag 300 may also include a membrane 326 covering the port 312. The membrane 326 may be configured to break when sufficient fluid pressure is applied to the membrane 326 to allow the fluid to flow through the port 312 into the mixing chamber 322. In this way, the membrane 326 may act as a frangible septum.

In some examples, the mixing chamber 322 may be sized and configured for holding a solid solute and for at least partially dissolving the solid solute in a liquid solvent (e.g., a fluid, purified water, etc.) entering the mixing chamber 322 through the port 312. The solid solute may partially fill the mixing chamber 322 before introduction of the fluid. For example, the solid solute may fill the mixing chamber 322 from the port 312 to a fill line 320.

When the liquid solvent (e.g., a fluid, purified water, etc.) is injected into the port 312, the membrane 326 may burst from the fluid pressure and allow the fluid to flow into the mixing chamber 322. For example, the fluid may be injected from the fluid source 166 through the valve 162 of FIG. 1. The injected fluid may flow into the mixing chamber 322 and mix with the solid solute. The mixing chamber 322 may include protrusions 304, 306, 308, 316, and 318 along the interior lateral sides of mixing chamber 322. Each of the protrusions 304, 306, 308, 316, and 318 may include a vertex extending laterally into the mixing chamber 322. The vertex of each protrusion may be located at a distance from the lower end of mixing chamber 322. For example, the vertex of the protrusion 304 may be located a distance D5 from the lower end of the mixing chamber 322, the vertex of the protrusion 306 may be located a distance D3 from the lower end of the mixing chamber 322, the vertex of the protrusion 308 may be located a distance D1 from the lower end of the mixing chamber 322, the vertex of the protrusion 316 may be located a distance D4 from the lower end of the mixing chamber 322, and the vertex of the protrusion 318 may be located a distance D2 from the lower end of the mixing chamber 322.

In some examples, as the fluid flows into the mixing chamber 322, the fluid may pass by the vertices of protrusions 304, 306, 308, 316, and 318, resulting in turbulence in the fluid flow. The turbulence may create vortices that may interact with each other and interact with the solid solute. The kinetic energy in the turbulent fluid flow may accelerate the homogenization (e.g., mixing) of the fluid solvent and the solid solute. The homogenization of the fluid and the solid solute may be based on factors including, without limitation, the pressure and/or flow rate of the fluid entering the port 312, the location of the protrusions 304, 306, 308, 316, and 318, the number of protrusions 304, 306, 308, 316, and 318, the shape of the protrusions 304, 306, 308, 316, and 318, the distance between the protrusions 304, 306, 308, 316, and 318, and the viscosity of the fluid. The interaction between the turbulent fluid and the solid solute may mix the fluid and the solid solute more thoroughly than a mixing chamber lacking the protrusions 304, 306, 308, 316, and 318.

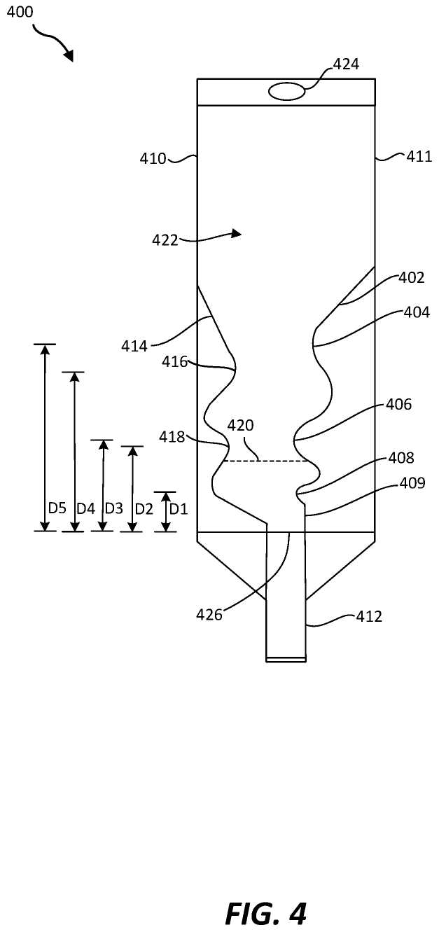

FIG. 4 is a detailed front view of a turbulent flow mixing bag 400, according to another embodiment of the present disclosure. The turbulent flow mixing bag 400 may be configured to mix materials (e.g., a liquid solvent and a solid solute) based on a turbulent flow of a fluid (e.g., a liquid) injected into the mixing bag 400. The mixing bag 400 may include a front wall, a back wall, and a mixing chamber 422 between the front wall and the back wall. The materials may be mixed in the mixing chamber 422 by the turbulent fluid motion of the injected fluid. The fluid motion may include chaotic and/or random changes in pressure and flow velocity within the mixing chamber 422 that may cause the injected fluid to mix with materials (e.g., a solid solute) within the mixing chamber 422. The mixing bag 400 may include a first sidewall 410 between the front wall and the back wall. The first sidewall 410 may define a first side of mixing chamber 422. The mixing bag 400 may also include a second sidewall 411 between the front wall and the back wall defining a second, opposite side of the mixing chamber 422. The front wall, back wall, first sidewall 410, and second sidewall 411 may include a polymer material (e.g., a medical grade polymer, polyvinyl chloride, polypropylene, copolyester ether, polyolefin, etc.). The mixing bag 400 may include a port 412 fluidically coupled to the mixing chamber 422 that is positioned and configured to provide fluid access to the mixing chamber 422 from a bottom of the mixing chamber 422. As will be described in further detail below, the first sidewall 410 and the second sidewall 411 may be shaped and configured to alternate a direction of fluid flow when fluid is introduced into the mixing chamber 422 through the port 412 to create the turbulent liquid flow for mixing the materials.

In some examples, the first sidewall 410 may include a plurality of straight sections 414, 419 that may be oriented at non-parallel angles to a longitudinal axis of the mixing chamber 422. Additionally or alternatively, the second sidewall 411 may include a plurality of straight sections 402, 409 that may be oriented at non-parallel angles to the longitudinal axis of the mixing chamber 422. Although FIG. 4 shows each of the first sidewall 410 and the second sidewall 411as including two straight sections of sidewalls, the present disclosure is not so limited and the mixing bag 400 may include any number of straight sections. As shown in FIG. 4, the angles of the straight sections 414, 419 of the first sidewall 410 may be different from the angles of the straight sections 402, 409 of the second sidewall 411. In some examples, each of the angles of the straight sections 414, 419 of the first sidewall 410 and the straight sections 402, 409 of the second sidewall 411 may be between about 20 degrees and about 80 degrees from the longitudinal axis. Different angles for each of the straight sections 402, 409, 414, 419 may contribute to the turbulent liquid flow for mixing the materials.

The mixing bag 400 may also include a hanger feature 424 at an end (e.g., a top) of the mixing bag 400 opposite the port 412. The hanger feature 424 may be configured for hanging the mixing bag 400 to support the mixing bag 400. For example, the hanger feature 424 may include a hole or a slit for hanging the mixing bag 400 on a support pole.

The mixing bag 400 may also include a membrane 426 covering the port 412. The membrane 426 may be configured to break when sufficient fluid pressure is applied to the membrane 426 to allow the fluid to flow through the port 412 into the mixing chamber 422.

In some examples, the mixing chamber 422 may be sized and configured for holding a solid solute and for at least partially dissolving the solid solute in a liquid solvent (e.g., a fluid, purified water, etc.) entering the mixing chamber 422 through the port 412. The solid solute may partially fill the mixing chamber 422 before introduction of the fluid. For example, the solid solute may fill the mixing chamber 422 from the port 412 to a fill line 420.

When the liquid solvent (e.g., a fluid, purified water, etc.) is injected into the port 412, the membrane 426 may burst from the fluid pressure and allow the fluid to flow into the mixing chamber 422. For example, the fluid may be injected from the fluid source 166 through the valve 162 of FIG. 1. The injected fluid may flow into the mixing chamber 422 and mix with the solid solute. The mixing chamber 422 may include protrusions 404, 406, 408, 416, and 418 along the interior lateral sides of the mixing chamber 422. Each of the protrusions 404, 406, 408, 416, and 418 may include a vertex extending laterally into mixing chamber 422. The vertex of each of the protrusions 404, 406, 408, 416, and 418 may be located at a distance from the lower end of the mixing chamber 422. For example, the vertex of the protrusion 404 may be located a distance D5 from the lower end of the mixing chamber 422, the vertex of the protrusion 406 may be located a distance D3 from the lower end of the mixing chamber 422, the vertex of the protrusion 408 may be located a distance D1 from the lower end of the mixing chamber 422, the vertex of the protrusion 416 may be located a distance D4 from the lower end of the mixing chamber 422, and the vertex of the protrusion 418 may be located a distance D2 from the lower end of the mixing chamber 422.

In some examples, the vertex of each protrusion may include a common endpoint of two segments of the sidewalls, as shown in FIGS. 1-3, for example. As shown in the example of FIG. 4, the protrusions 404, 406, 408, 416, and 418 may exhibit a rounded shape. The protrusions 404, 406, 408, 416, and 418 may each have the same radius of curvature or a different radius of curvature. For example, the radii of curvature for the protrusions 404, 406, 408, and 418 may respectively increase as the distance increases from the lower end of the mixing chamber 422 at which the protrusions 404, 406, 408, and 418 are located. In additional examples, one or more of the protrusions 404, 406, 408, 416, and 418 may have a non-uniform radius of curvature. Different curvatures for each of the protrusions 404, 406, 408, 416, and 418 may contribute to an increased turbulence for improved mixing of the materials.

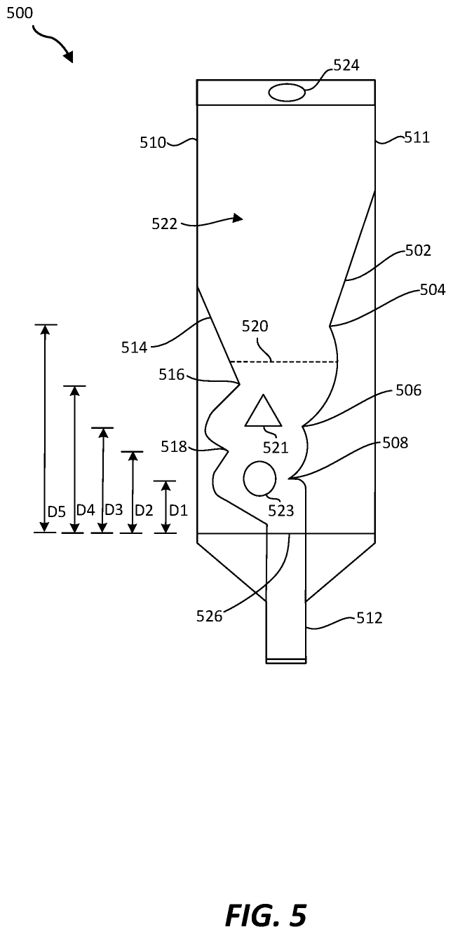

FIG. 5 is a detailed front view of a turbulent flow mixing bag 500, according to another embodiment of the present disclosure. The turbulent flow mixing bag 500 may be configured to mix materials (e.g., a liquid solvent with a solid solute) based on a turbulent flow of a fluid (e.g., a liquid) injected into the mixing bag 500. The mixing bag 500 may include a front wall, a back wall, and a mixing chamber 522 between the front wall and the back wall. The materials may be mixed in the mixing chamber 522 by the turbulent fluid motion of the injected fluid. The fluid motion may include chaotic and/or random changes in pressure and flow velocity within the mixing chamber 522 that may cause the injected fluid to mix with materials (e.g., a solid solute) within the mixing chamber 522. The mixing bag 500 may include a first sidewall 510 between the front wall and the back wall and defining a first side of the mixing chamber 522. The mixing bag 500 may also include a second sidewall 511 between the front wall and the back wall defining a second, opposite side of the mixing chamber 522. The front wall, back wall, first sidewall 510, and second sidewall 511 may include a polymer material (e.g., a medical grade polymer, polyvinyl chloride, polypropylene, copolyester ether, polyolefin, etc.). The mixing bag 500 may include a port 512 fluidically coupled to the mixing chamber 522 that is positioned and configured to provide fluid access to the mixing chamber 522 from a bottom of the mixing chamber 522. As will be described in further detail below, the first sidewall 510 and the second sidewall 511 may be shaped and configured to alternate a direction of fluid flow when fluid is introduced into the mixing chamber 522 through the port 512 to create the turbulent liquid flow for mixing the materials.

In some examples, the first sidewall 510 may include a plurality of straight sections 514, 519 that may be oriented at non-parallel angles to a longitudinal axis of mixing chamber 522. Additionally or alternatively, the second sidewall 511 may include a plurality of straight sections 502, 509 that may be oriented at non-parallel angles to a longitudinal axis of mixing chamber 522. Although FIG. 5 shows each of the first sidewall 510 and the second sidewall 511 including two straight sections of sidewalls, the present disclosure is not so limited and the mixing bag 500 may include any number of straight sections. As shown in FIG. 5, the angles of the straight sections 514, 519 of first sidewall 510 may be different from the angles of the straight sections 502, 509 of the second sidewall 511. In some examples, each of the angles of the straight sections 514, 519 of the first sidewall 510 and the straight sections 502, 509 of the second sidewall 511 may be between about 20 degrees and about 80 degrees from the longitudinal axis. Different angles for each of the straight sections 502, 509, 514, 519 may contribute to the turbulent liquid flow for mixing the materials.

The mixing bag 500 may also include a hanger feature 524 at an end (e.g., a top) of the mixing bag 500 opposite the port 512. The hanger feature 524 may be configured for hanging the mixing bag 500 to support the mixing bag 500. For example, the hanger feature 524 may include a hole or slit for hanging the mixing bag 500 on a support pole.

The mixing bag 500 may also include a membrane 526 covering the port 512. The membrane 526 may be configured to break when a sufficient fluid pressure is applied to the membrane 526 to allow the fluid to flow through the port 512 into the mixing chamber 522.

In some examples, the mixing chamber 522 may be sized and configured for holding a solid solute and for at least partially dissolving the solid solute in a liquid solvent (e.g., a fluid, purified water, etc.) entering the mixing chamber 522 through the port 512. The solid solute may partially fill the mixing chamber 522 before introduction of the fluid. For example, the solid solute may fill the mixing chamber 522 from the port 512 to a fill line 520.

When the liquid solvent (e.g., a fluid, purified water, etc.) is injected into the port 512, the membrane 526 may burst from the fluid pressure and allow the fluid to flow into the mixing chamber 522. For example, the fluid may be injected from the fluid source 166 through valve 162 of FIG. 1. The injected fluid may flow into the mixing chamber 522 and mix with the solid solute. The mixing chamber 522 may include protrusions 504, 506, 508, 516, and 518 along the interior lateral sides of the mixing chamber 522. Each of the protrusions 504, 506, 508, and 518 may include a vertex extending laterally into the mixing chamber 522. The vertex of each of the protrusions 504, 506, 508, 516, and 518 may be located at a distance from the lower end of the mixing chamber 522. For example, the vertex of the protrusion 504 may be located a distance D5 from the lower end of the mixing chamber 522, the vertex of the protrusion 506 may be located a distance D3 from the lower end of the mixing chamber 522, the vertex of the protrusion 508 may be located a distance D1 from the lower end of the mixing chamber 522, the vertex of the protrusion 516 may be located a distance D4 from the lower end of the mixing chamber 522, and the vertex of the protrusion 518 may be located a distance D2 from the lower end of the mixing chamber 522.

In some examples, the vertex of each protrusion may include a common endpoint of two portions of the sidewalls, as shown in FIGS. 1-3. In some examples, the protrusions 504, 506, 508, 516, and 518 may have any shape that results in the turbulent flow. Additionally or alternatively, the mixing bag 500 may include barriers 521, 523. The barriers 521, 523 may be positioned within the mixing chamber 522 and between the sidewalls of the mixing chamber 522. When the fluid passes by the vertices of the barriers 521, 523, turbulence in the fluid may be induced by interaction with the barriers 521, 523. Any number of barriers 521, 523 may be positioned and configured within the mixing chamber 522. In FIG. 5, the barrier 521 has a triangle shape and the barrier 523 has a circular shape. However, the present disclosure is not so limited and the barriers 521, 523 may have any shape for inducing turbulence. The barriers 521, 523 may contribute to the turbulence to enhance mixing of the materials within the mixing chamber 522.

In some examples, as the fluid flows into mixing chamber 522, the fluid may pass by the vertices of the protrusions 504, 506, 508, 516, and 518 and by the barriers 521 and 523 to create turbulence in the fluid flow. The turbulence in the fluid flow may create vortices that may interact with each other and that may interact with the solid solute. The kinetic energy in the turbulent fluid flow may accelerate the homogenization (e.g., mixing) of the fluid and the solid solute. The homogenization of the fluid and the solid solute may be based on factors including, without limitation, the pressure and/or flow rate of the fluid entering the port 512, the location of the protrusions 504, 506, 508, 516, and 518, the number of the protrusions 504, 506, 508, 516, and 518, the location of the barriers 521 and 523, the number of the barriers 521 and 523, the shape of the protrusions 504, 506, 508, 516, and 518, the shape of the barriers 521 and 523, the distance between the protrusions 504, 506, 508, 516, and 518, the distance between the barriers 521 and 523, the distance between the protrusions 504, 506, 508, 516, and 518 and the barriers 521 and 523, and the viscosity of the fluid. The interaction between the turbulent fluid flow and the solid solute may mix the fluid and the solid solute more thoroughly than a mixing chamber without protrusions and/or barriers.

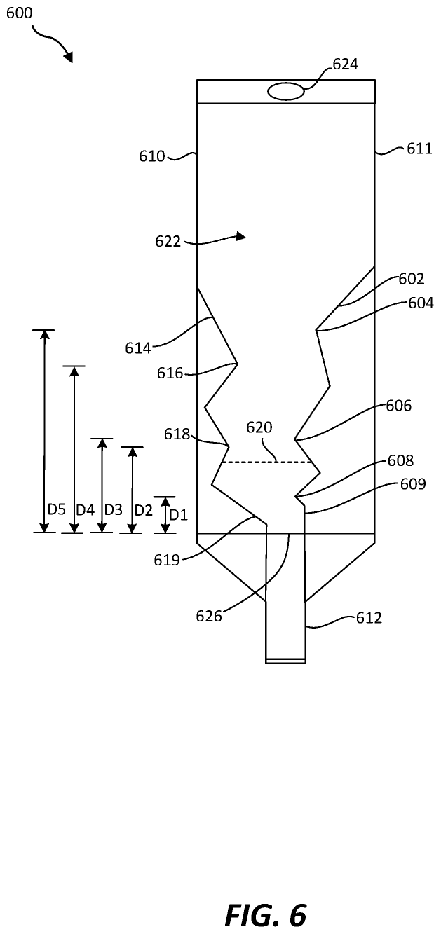

FIG. 6 is a detailed front view of a turbulent flow mixing bag 600, according to another embodiment of the present disclosure. The turbulent flow mixing bag 600 may be configured to mix materials (e.g., a liquid solvent with a solid solute) based on a turbulent flow of a fluid (e.g., a liquid) injected into the mixing bag 600. The mixing bag 600 may include a front wall, a back wall, and a mixing chamber 622 between the front wall and the back wall. The materials may be mixed in the mixing chamber 622 by the turbulent fluid motion of the injected fluid. The fluid motion may include chaotic and/or random changes in pressure and flow velocity within the mixing chamber 622 that may cause the injected fluid to mix with materials (e.g., a dialysate) within the mixing chamber 622.

The mixing bag 600 may include a first sidewall 610 between the front wall and the back wall, which may define a first side of the mixing chamber 622. The mixing bag 600 may also include a second sidewall 611 between the front wall and the back wall defining a second, opposite side of the mixing chamber 622. The front wall, back wall, first sidewall 610, and second sidewall 611 may include a polymer material (e.g., a medical grade polymer, polyvinyl chloride, polypropylene, copolyester ether, polyolefin, etc.). The mixing bag 600 may include a port 612 fluidically coupled to the mixing chamber 622. The port 612 may be positioned and configured to provide fluid access to the mixing chamber 622 from a bottom of the mixing chamber 622. As will be described in further detail below, the first sidewall 610 and second sidewall 611 may be shaped and configured to alternate a direction of fluid flow when fluid is introduced into the mixing chamber 622 through port 612 and/or when fluid is withdrawn from the mixing chamber 622 through the port 612 to create the turbulent flow for mixing the materials.

In some examples, the first sidewall 610 may include a plurality of straight sections 614, 619 that may be oriented at non-parallel angles to a longitudinal axis of the mixing chamber 622. Additionally or alternatively, the second sidewall 611 may include a plurality of straight sections 602, 609 that may be oriented at non-parallel angles to the longitudinal axis of the mixing chamber 622. As shown in FIG. 6, each of the first sidewall 610 and the second sidewall 611 may be defined by straight sections 602, 609, 614, 619 (e.g., without any curved sections). The presence of the straight sections 602, 609, 614, 619 without any curved sections of the sidewalls 610, 611 may, in some embodiments, result in increased turbulence in the mixing chamber 622. In some examples, the angles of the straight sections 614, 619 of the first sidewall 610 may be different from the angles of the straight sections 602, 609 of second sidewall 611. In some examples, each of the angles of the straight sections 614, 619 of the first sidewall 610 and the straight sections 602, 609 of the second sidewall 611 may be between about 20 degrees and about 80 degrees from the longitudinal axis. Different angles for each of the straight sidewalls 602, 609, 614, 619 may contribute to the turbulent liquid flow for mixing the materials.

The mixing bag 600 may also include a hanger feature 624 at an end (e.g., a top) of the mixing bag 600 opposite the port 612. The hanger feature 624 may be configured for hanging the mixing bag 600 to support the mixing bag 600. For example, the hanger feature 624 may include a hole or a slit for hanging the mixing bag 600 on a support pole.

The mixing bag 600 may also include a membrane 626 covering the port 612. The membrane 626 may be configured to break when sufficient fluid pressure is applied to the membrane 626 to allow the fluid to flow through the port 612 into the mixing chamber 622.

In some examples, the mixing chamber 622 may be sized and configured for holding a solid solute and for at least partially dissolving the solid solute in a liquid solvent (e.g., a fluid, purified water, etc.) entering the mixing chamber 622 through the port 612. The solid solute may partially fill the mixing chamber 622 before introduction of the fluid. For example, the solid solute may fill the mixing chamber 622 from the port 612 to a fill line 620.

When the liquid solvent (e.g., a fluid, purified water, etc.) is injected into the port 612, the membrane 626 may burst from the fluid pressure and allow the fluid to flow into the mixing chamber 622. For example, the fluid may be injected from the fluid source 166 through the valve 162 of FIG. 1. The injected fluid may flow into the mixing chamber 622 and mix with the solid solute. The mixing chamber 622 may include protrusions 604, 606, 608, 616, and 618 along the interior lateral sides of the mixing chamber 622. Each of the protrusions 604, 606, 608, 616, and 618 may include a vertex extending laterally into the mixing chamber 622. The vertex of each of the protrusion 604, 606, 608, 616, and 618 may be located at a distance from the lower end of the mixing chamber 622. For example, the vertex of the protrusion 604 may be located a distance D5 from the base of the mixing chamber 622, the vertex of the protrusion 606 may be located a distance D3 from the base of the mixing chamber 622, the vertex of the protrusion 608 may be located a distance D1 from the base of the mixing chamber 622, the vertex of the protrusion 616 may be located a distance D4 from the base of the mixing chamber 622, and the vertex of the protrusion 618 may be located a distance D2 from the base of the mixing chamber 622. In some examples, the vertex of each protrusion may include a common endpoint of two segments of the sidewalls 610, 611, as shown in FIG. 6. Each segment of the sidewalls 610, 611 may be a straight segment and the common endpoint of each two adjacent segments may be a corner or a point where the straight segments meet. The included angle at each vertex of the protrusions 604, 606, 608, 616, and 618 may contribute to the turbulent liquid flow for mixing the materials.

In some examples, as the fluid flows into the mixing chamber 622, at least some of the fluid may pass by the vertices of protrusions 604, 606, 608, 616, and 618 creating turbulence in the fluid flow. The turbulence in the fluid flow may create vortices that may interact with each other and that may interact with the solid solute. The kinetic energy in the turbulent fluid flow may accelerate the homogenization (e.g., mixing) of the fluid and the solid solute. The homogenization of the fluid and the solid solute may be based on factors including, without limitation, the pressure and/or flow rate of the fluid entering the port 612, the location of the protrusions 604, 606, 608, 616, and 618, the number of the protrusions 604, 606, 608, 616, and 618, the shape of the protrusions 604, 606, 608, 616, and 618, the distance between the protrusions 604, 606, 608, 616, and 618, and the viscosity of the fluid. The interaction between the turbulent fluid flow and the solid solute may mix the fluid and the solid solute more thoroughly than a mixing chamber without protrusions.

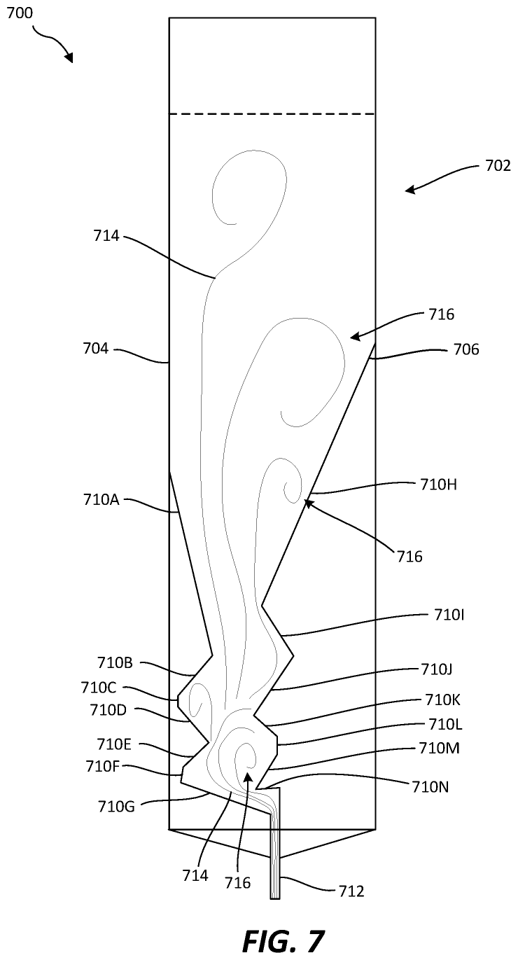

FIG. 7 is an illustration of a fluid flow model 700 showing turbulence in a mixing bag 702, according to at least one embodiment of the present disclosure. In some respects, the mixing bag 702 may be similar to the mixing bags 200, 300, 400, 500, 600 described above. For example, the mixing bag 702 may include a front wall, a back wall, a first sidewall 704, and a second sidewall 706. The first sidewall 704 may define a first lateral side of a mixing chamber 708 within the mixing bag 702, and the second sidewall 706 may define a second, opposite lateral side of the mixing chamber 708. Each of the first sidewall 704 and the second sidewall 706 may include sidewall segments 710A-710N. At least some of the sidewall segments 710A-710N may be at non-parallel angles to a longitudinal axis of the mixing bag 702. A port 712 may be located and configured to introduce a fluid (e.g., a liquid solvent) into the mixing chamber 708 from a bottom of the mixing chamber 708. The port 712 may also be used to withdraw fluid (e.g., a dialysate or a component thereof including a solution of the liquid solvent and a solid solute), such as for use in a hemodialysis operation. The sidewall segments 710A-710N may define protrusions into the mixing chamber 708, which may be configured for inducing turbulence when fluid is introduced into and/or withdrawn from the mixing chamber 708 through the port 712.

As illustrated in FIG. 7 by flowlines 714, fluid flowing into the mixing chamber 708 through the port 712 and mixing with a solid solute within the mixing chamber 708 may follow one or more tortuous routes through the mixing chamber 708. One or more flow vortices 716 may develop as the fluid passes along the sidewall segments 710A-710N and around the protrusions in the sidewalls 704, 706. The presence of these flow vortices 716 may improve mixing of the liquid solvent with the solid solute to encourage and speed up dissolution of the solid solute in the liquid solvent.

Accordingly, the present disclosure includes turbulent flow mixing bags for mixing a solid solute with a liquid solvent, such as to form a dialysate solution (or a component thereof), and related hemodialysis systems. Embodiments of the present disclosure may provide one or more improvements over conventional methods and devices for mixing dialysate solution for hemodialysis. For example, the turbulent flow mixing bag of the present disclosure may include protrusions and vertices along the sidewalls of the mixing bag to create a turbulent flow of injected liquid for mixing with a solid solute.

The following example embodiments are also included in the present disclosure.

Example 1: A bag for mixing materials, which may include a front wall, a back wall, a mixing chamber between the front wall and the back wall, a first sidewall between the front wall and the back wall and defining a first side of the mixing chamber, a second sidewall between the front wall and the back wall defining a second, opposite side of the mixing chamber, and a port positioned to provide fluid access to the mixing chamber from a bottom of the mixing chamber, wherein the first sidewall and the second sidewall are shaped to alternate a direction of fluid flow when fluid is introduced into the mixing chamber through the port.

Example 2: The bag for mixing materials of Example 1, wherein each of the first sidewall and the second sidewall comprises at least three distinct straight sections.

Example 3: The bag for mixing materials of Example 1 or Example 2, wherein each of the first sidewall and the second sidewall comprises at least three distinct straight sections.

Example 4: The bag for mixing materials of any of Examples 1 through 3, wherein the angles of the straight sections of the first sidewall are different from angles of the straight sections of the second sidewall.

Example 5: The bag for mixing materials of any of Examples 1 through 4, wherein each of the angles of the straight sections is between about 20 degrees and about 80 degrees from the longitudinal axis.

Example 6: The bag of Example 5, wherein at least some of the angles of the straight sections respectively alternate from between about 20 degrees and about 80 degrees in a counterclockwise direction from the longitudinal axis to between about 20 degrees and about 80 degrees in a clockwise direction from the longitudinal axis.

Example 7: The bag of Example 5 or Example 6, wherein each of the angles of the straight sections is between about 30 degrees and about 60 degrees from the longitudinal axis.

Example 8: The bag of any of Examples 5 through 7, wherein at least one of the straight sections of the first sidewall has an angle of between about 35 degrees and about 55 degrees in a clockwise direction from the longitudinal axis and at least one other straight section of the first sidewall has an angle of between about 35 degrees and about 55 degrees in a counterclockwise direction from the longitudinal axis.

Example 9: The bag of any of Examples 2 through 8, wherein at least one of the first sidewall or the second sidewall comprises an additional straight section that is oriented parallel to the longitudinal axis of the mixing chamber.

Example 10: The bag for mixing materials of any of Examples 1 through 9, wherein a lateral width between the first sidewall and the second sidewall and perpendicular to a longitudinal axis of the mixing chamber increases as a distance from the port increases.

Example 11: The bag for mixing materials of any of Examples 1 through 10, wherein a lateral width between the first sidewall and the second sidewall and a perpendicular to a longitudinal axis of the mixing chamber changes between an increasing width and decreasing width as a distance from the port increases.

Example 12: The bag for mixing materials of any of Examples 1 through 11, further comprising a hanger feature at an end of the mixing chamber opposite the port, the hanger feature configured for hanging the bag to support the bag.

Example 13: The bag for mixing materials of any of Examples 1 through 12, further comprising a membrane covering the port, wherein the membrane is configured to break to allow fluid to flow through the port into the mixing chamber.

Example 14: The bag for mixing materials of any of Examples 1 through 13, wherein the front wall, the back wall, the first sidewall, and the second sidewall comprise a polymer material.

Example 15: The bag for mixing materials of any of Examples 1 through 14, wherein the first sidewall and the second sidewall comprise portions of the front wall and the back wall that are sealed to each other.

Example 16: A bag for mixing materials, may include a front wall, a back wall, a mixing chamber between the front wall and the back wall, a port positioned to provide fluid access to the mixing chamber from a bottom of the mixing chamber, and protrusions into the mixing chamber configured to induce turbulence in fluid flowing into the mixing chamber through the port.

Example 17: The bag for mixing materials of Example 16, wherein the mixing chamber is sized and configured for holding a solid solute and for at least partially dissolving the solid solute in a liquid solvent entering the mixing chamber through the port.

Example 18: The bag for mixing materials of Example 16 or Example 17, wherein the protrusions are defined by sections of the front wall and the back wall that are sealed to each other.

Example 19: The bag for mixing materials of any of Examples 16 through 18, wherein each of the protrusions comprises a vertex extending laterally into the mixing chamber.

Example 20: The bag for mixing materials of any of Examples 16 through 19, wherein the protrusions comprise at least one first protrusion on a first lateral side of the mixing chamber and at least one second protrusion on a second, opposite lateral side of the mixing chamber.

Example 21: The bag for mixing materials of any of Examples 16 through 20, wherein the at least one first protrusion comprises a plurality of first protrusions and the at least one second protrusion comprises a plurality of second protrusions.

Example 22: A hemodialysis system, which may include a dialyzer configured to withdraw at least one product from an intended patient's blood, the dialyzer comprising a dialyzer blood inlet, a dialyzer blood outlet, a dialysate inlet, and a dialysate outlet, and a dialysate production system configured to mix a liquid solvent with a solid solute to form a dialysate solution to provide to the dialyzer, wherein the dialysate production system comprises a mixing chamber for mixing a solid solute in a liquid solvent, the mixing chamber comprising a single port for fluid inlet and fluid outlet, and sidewalls shaped to induce fluid flowing in the mixing chamber from the single port to alternate directions within the mixing chamber.

Example 23: The hemodialysis system of Example 22, wherein the dialysate production system further comprises a fluid source and a fluid conduit for flowing fluid to the mixing chamber via the single port.

Example 24: The hemodialysis system of Example 22 or Example 23, wherein the each of the sidewalls of the mixing chamber alternates between being angled toward a longitudinal axis of the mixing chamber and away from the longitudinal axis of the mixing chamber.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A bag for mixing materials, comprising:
a front wall;
a back wall;
a mixing chamber between the front wall and the back wall;
a first sidewall between the front wall and the back wall and defining a first side of the mixing chamber;
a second sidewall between the front wall and the back wall defining a second, opposite side of the mixing chamber; and
a port positioned to provide fluid access to the mixing chamber from a bottom of the mixing chamber,
wherein the first sidewall and the second sidewall are shaped to alternate a direction of fluid flow when fluid is introduced into the mixing chamber through the port.

2. The bag of claim 1, wherein each of the first sidewall and the second sidewall comprises a plurality of straight sections oriented at non-parallel angles to a longitudinal axis of the mixing chamber.

3. The bag of claim 2, wherein each of the first sidewall and the second sidewall comprises at least three distinct straight sections.

4. The bag of claim 2, wherein the angles of the straight sections of the first sidewall are different from angles of the straight sections of the second sidewall.

5. The bag of claim 2, wherein each of the angles of the straight sections is between about 20 degrees and about 80 degrees from the longitudinal axis.

6. The bag of claim 5, wherein at least some of the angles of the straight sections respectively alternate from between about 20 degrees and about 80 degrees in a counterclockwise direction from the longitudinal axis to between about 20 degrees and about 80 degrees in a clockwise direction from the longitudinal axis.

7. The bag of claim 5, wherein each of the angles of the straight sections is between about 30 degrees and about 60 degrees from the longitudinal axis.

8. The bag of claim 5, wherein at least one of the straight sections of the first sidewall has an angle of between about 35 degrees and about 55 degrees in a clockwise direction from the longitudinal axis and at least one other straight section of the first sidewall has an angle of between about 35 degrees and about 55 degrees in a counterclockwise direction from the longitudinal axis.

9. The bag of claim 2, wherein at least one of the first sidewall or the second sidewall comprises an additional straight section that is oriented parallel to the longitudinal axis of the mixing chamber.

10. The bag of claim 1, wherein a lateral width between the first sidewall and the second sidewall and perpendicular to a longitudinal axis of the mixing chamber increases as a distance from the port increases.

11. The bag of claim 1, wherein a lateral width between the first sidewall and the second sidewall and perpendicular to a longitudinal axis of the mixing chamber changes between an increasing width and decreasing width as a distance from the port increases.

12. The bag of claim 1, further comprising a hanger feature at an end of the mixing chamber opposite the port, the hanger feature configured for hanging the bag to support the bag.

13. The bag of claim 1, further comprising a membrane covering the port, wherein the membrane is configured to break to allow fluid to flow through the port into the mixing chamber.

14. The bag of claim 1, wherein the front wall, the back wall, the first sidewall, and the second sidewall comprise a polymer material.

15. The bag of claim 1, wherein the first sidewall and the second sidewall comprise portions of the front wall and the back wall that are sealed to each other.

16. A bag for mixing materials, comprising:
a front wall;
a back wall;
a mixing chamber between the front wall and the back wall;
a port positioned to provide fluid access to the mixing chamber from a bottom of the mixing chamber; and
protrusions into the mixing chamber configured to induce turbulence in fluid flowing into the mixing chamber through the port.

17. The bag of claim 16, wherein the mixing chamber is sized and configured for holding a solid solute and for at least partially dissolving the solid solute in a liquid solvent entering the mixing chamber through the port.

18. The bag of claim 16, wherein the protrusions are defined by sections of the front wall and the back wall that are sealed to each other.

19. The bag of claim 16, wherein each of the protrusions comprises a vertex extending laterally into the mixing chamber.

20. The bag of claim 16, wherein the protrusions comprise at least one first protrusion on a first lateral side of the mixing chamber and at least one second protrusion on a second, opposite lateral side of the mixing chamber.
